(19) United States
(12) Patent Application Publication
(10) Pub. No.: US 2019/0374368 A1
(43) Pub. Date: Dec. 12, 2019

(54) DIAGNOSTIC METHOD AND NOSE CLIP FOR SELECTING TREATMENT FOR LATERAL WALL WEAKNESS/NASAL VALVE COLLAPSE IN A NOSE

(71) Applicant: Dean M. Toriumi, Riverside, IL (US)

(72) Inventor: Dean M. Toriumi, Riverside, IL (US)

(21) Appl. No.: 16/006,545

(22) Filed: Jun. 12, 2018

(51) Int. Cl. A61F 5/08; A61B 5/00; A61B 5/08

(52) U.S. Cl. CPC A61F 5/08; A61B 5/4836; A61B 5/08; A61B 5/6819

(57) ABSTRACT

A method for selecting an appropriate treatment for a patient with nasal valve lateral wall collapse is provided includes forming a plurality of nose clips from a semi-ridge material. Each nose clip has an outer support section configured to overlay a contour of an outside surface of a nose, an inner support section configured to be placed inside the nasal cavity of the nose and to support the lateral wall of the nasal cavity towards the outer support section, and a bridge section that connects the outer support to the inner support section. First and second nose clips are configured to provide first and second amounts of support between the inner and outer support sections, respectively. The first nose clip is inserted into a nasal cavity of the patient. A determination is made as to whether insertion of the first nose clip sufficiently reduces nasal valve lateral wall collapse in the patient. If so, the method includes determining that a first treatment will effectively reduce nasal valve lateral wall collapse in the patient. Otherwise, the method includes inserting the second nose clip into the nasal cavity of the patient and determining whether the second nose clip sufficiently reduces nasal valve lateral wall collapse. If so, the method includes determining that a second treatment will effectively reduce nasal valve lateral wall collapse in the patient.

### BACKGROUND

### Field of Application

The present application is related to medical devices. More specifically, the present application is related to a diagnostic method and nose clip used in the method for selecting treatment for lateral wall weakness and nasal valve collapse/obstruction in a nose.

### Discussion

When some patients breathe in through their nose, the lateral wall and/or nasal valve of their nose collapses medially causing nasal obstruction. This disorder can be improved if patients wear, for example, Breathe Right Strips® or place nose cones in their nose.

Surgery may be an option in some cases. For example, cartilage grafts such as alar batten grafts or lateral crural strut grafts that act to support the lateral wall of the nose may be placed in the nose to improve nasal breathing. Surgery will not work to correct the disorder in all cases, however. Thus, proper diagnosis to determine those cases where the patient would benefit from surgery is important. If improperly diagnosed, patients may undergo surgery with no improvement to their breathing, putting the patient through unnecessary risk and unnecessary cost for the surgery.

The most common method for diagnosing nasal valve collapse or more specifically lateral wall weakness is the so-called modified Cottle maneuver. This diagnostic method involves inserting a probe against the interior lateral wall of the patient's nose to support the lateral side wall during breathing. The patient then indicates to the clinician whether breathing is improved.

A key to successful use of this diagnostic method is to avoid moving the lateral wall out too much. If moved out too much, the patient may experience an improvement to breathing that is unattainable by placing cartilage grafts into the nose.

Unfortunately, many clinicians will move the lateral wall out too much creating a false positive test that falsely identifies a patient with nasal valve collapse that would be effectively treated with grafting. This inter-clinician variability is a significant problem that creates false hope for patients and results in wasted healthcare costs with unnecessary risk.

### SUMMARY

In a first aspect, a method for selecting an appropriate treatment for a patient with nasal valve lateral wall collapse is provided. The method includes forming a plurality of nose clips from a variety of materials. Each nose clip has an outer support section configured to overlay a contour of an outside surface of a nose, an inner support section configured to be placed inside the nasal cavity of the nose and to support the lateral wall of the nasal cavity towards the outer support section, and a bridge section that connects the outer support to the inner support section. First and second nose clips are configured to provide first and second amounts of support between the inner and outer support sections, respectively. The first nose clip is inserted into a nasal cavity of the patient. A determination is made as to whether insertion of the first nose clip sufficiently reduces nasal valve lateral wall collapse in the patient. If so, the method includes determining that a first treatment will effectively reduce nasal valve lateral wall collapse in the patient. Otherwise, the method includes inserting the second nose clip into the nasal cavity of the patient and determining whether insertion of the second nose clip sufficiently reduces nasal valve lateral wall collapse. If the second nose clip sufficiently reduces nasal valve lateral wall collapse, the method includes determining that a second treatment will effectively reduce nasal valve lateral wall collapse in the patient. In situations where a patient may be interested in a non-surgical option for management, a third wire-like diagnostic nose clip can be placed on the lateral wall extending up toward the nasal bones. If the patient experiences improvement, then a minimally invasive vertically oriented implant may be a good option to correct the patient's nasal valve lateral wall collapse.

In a second aspect, a diagnostic kit for diagnosing lateral wall weakness and/or nasal valve collapse/obstruction of a patient's nose includes a plurality of nose clips formed from a stiff material. Each nose clip has an outer support section configured to overlay a contour of an outside surface of a nose, an inner support section configured to be placed inside the nasal cavity of the nose and to support the lateral wall of the nasal cavity outward towards the outer support section, and a bridge section that connects the outer support to the inner support section. The first and second nose clips are configured to provide a first and second amount of support between the inner and outer support sections, respectively. The second amount of support is greater than the first amount of support and has a different shape and effect. If a minimally invasive approach is a consideration, the wire-like nose clip can be placed on the lateral wall of the nose to assess if the patient would be a candidate. Each nose clip is associated with one of a plurality of medical procedures for correcting lateral wall weakness. During diagnoses, nose clips are inserted into the patient's nose to identify a nose clip that provides a discernable improvement in breathing. This in turn facilitates determining a medical procedure for the correcting lateral wall weakness and or nasal valve collapse/obstruction of the patient's nose.

### BRIEF DESCRIPTION OF THE DRAWINGS

FIG. 1A is a front perspective view of an exemplary nose clip for diagnosing lateral wall weakness;

FIG. 1B is a side view thereof;

FIGS. 1C and 1D illustrate the nose clip when inserted into a patient's nose; and

FIGS. 2A-2C show nose clips of different configurations in a nose;

FIG. 3 illustrates a method that utilizes one or more nose clip embodiments to determine whether a patient is a suitable candidate for a surgical procedure to correct lateral weakness in the nose.

### DETAILED DESCRIPTION

The embodiments below describe a nose clip and a diagnostic method that utilizes the nose clip to diagnose lateral wall weakness and or nasal valve collapse/obstruction in the nose of a patient and for determining an appropriate treatment for treating the lateral wall weakness. Generally, an exemplary nose clip is provided in different sizes to accommodate noses of different sizes. The nose clips are also provided in different widths to facilitate determining the appropriate treatment.

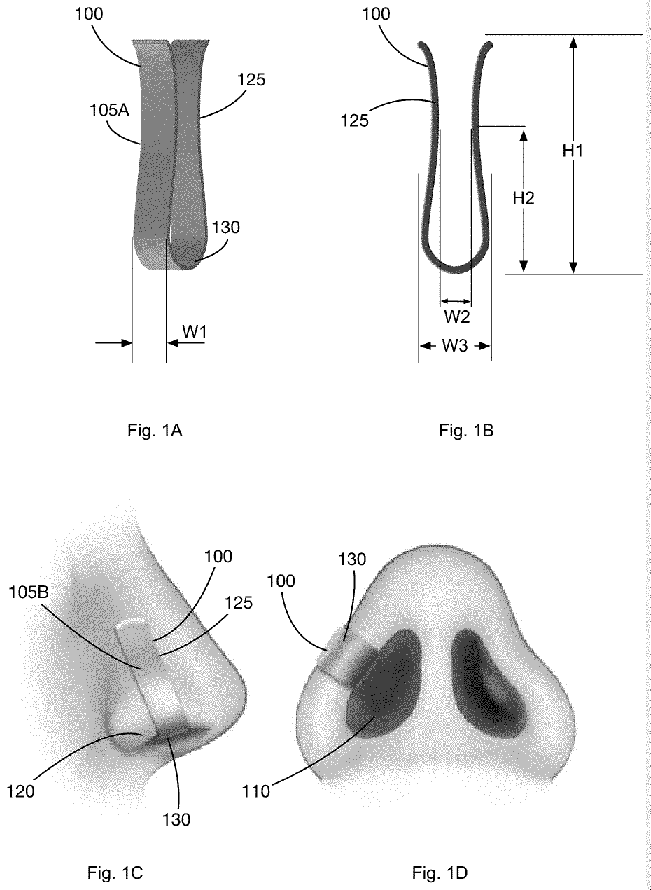

FIG. 1A is a front perspective view of an exemplary nose clip 100 for diagnosing lateral wall weakness and/or nasal valve collapse/obstruction. The nose clip 100 comprises a ribbon-shaped material that defines an inner support section 105A, an outer support section 105B, and a bridge section 130 that connects the inner and outer support sections 105A, 105B to one another. FIGS. 1C and 1D show the inner support section 105A positioned within the nostril, where the bridge section 130 wraps around the alar rim 120, and the outer support section 105B is positioned outside of the nostril.

The inner support section 105A and outer support section 105B may have similar shapes and sizes or may be different. As illustrated in the side view of FIG. 1B, the bridge section 130 may have a bulbous shape configured to wrap comfortably around the alar rim 130 of the nose. The inner and outer support sections 105A, 105B may gradually curve inwards towards each other and come closest to one another near a middle region 125 of the respective support sections 105A, 105B that, when placed on a patient, may correspond generally to the position of the alar crease. Ends of the support sections 105A, 105B may curve away from one another as shown. The curved end features facilitate smooth insertion of the nose clip into the nostril and minimize or eliminate scraping of the tissue within the nasal cavity during insertion of the nose clip that might otherwise occur.

The nose clip 100 described above may be provided in different configurations that include various sizes to accommodate noses of different sizes. Different configurations are also provided to facilitate diagnosing lateral wall weakness or nasal valve collapse/obstruction and for selecting an appropriate treatment for the same. For example, the nose clip may be provided in configurations having different heights, H1, H2, and widths, W2, and W3, to accommodate noses of different sizes. For example, in one exemplary implementation, height H1 may be 1.2 cm, 1.5 cm or 2 cm to accommodate different size noses. Width W3 may be sized to accommodate alar rims of difference sizes. Width W2 may be sized to secure the nose clip 100 within the nasal cavity 110 while supporting the lateral wall of the nose, without affecting the shape of the lateral wall.

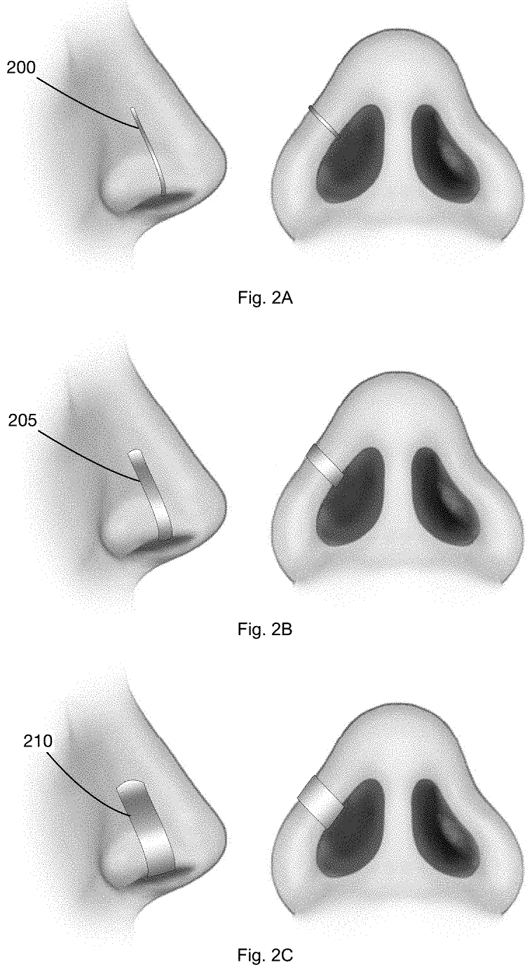

As illustrated in FIGS. 2A-2C, the width, W1, may be selected to provide different levels of lateral wall support during patient breathing. For example, a first nose clip 200 may have a width, W1, sized to provide support between the nasal bone and supra-alar groove. In an exemplary implementation, the first nose clip 200 may have a wire like form and have a width, W1, of about 1 mm to provide a linear area of support that is less than the broader nose clips (205, 210). A second nose clip 205 may have a width, W1, sized to provide support along the area along the supra-alar groove. In an exemplary implementation, the second nose clip 205 may have a width, W1, of about 5 mm. A third nose clip 210 may have a width, W1, sized to provide support along the lateral wall between the nasal bone and nostril margin (entire lateral wall of nose). In an exemplary implementation, the third nose clip 210 may have a width, W1, of about 10 mm. Together, the three nose clips described above facilitate diagnosing lateral wall collapse and nasal valve collapse/obstruction and for selecting an appropriate treatment. Selection of the treatment is described in more detail below. Determination as to the association between treatment/procedures and different nose clip configurations may have been previously determined via clinical trials.

In addition to the configurations above, the rigidity of the nose clips may be different. For example, the nose clips may be formed from materials having different rigidities (e.g., plastic, metal, etc.). The thickness and/or diameter of the material from which the nose clip is formed may be different. In some embodiments, the nose clip material may be encased in plastic or rubber (e.g., silicone rubber) to improve securing the nose clip 100 to the nose and to provide additional comfort.

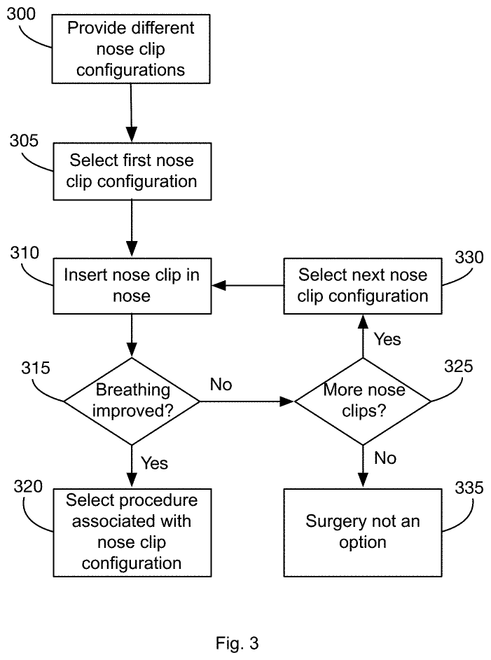

FIG. 3 illustrates a diagnostic method for diagnosing lateral wall weakness and for selecting an appropriate treatment for the lateral wall weakness.

In a first operation 300, different nose clip configurations, such as the first, second, and third nose clips described above, may be provided. Each nose clip may be configured to provide a different amount of lateral wall support with minimal, if any, movement to the lateral wall.

In a next operation, 305, the first nose clip having the first configuration may be selected and at operation 310, the nose clip may be inserted in the patient's nose. During insertion, the clinician may insert the nose clip and determine whether any appreciable movement to the lateral wall of the nose occurs. If any appreciable movement is detected, then a different nose clip that causes minimal movement is selected.

At operation 315, the patient may be asked to rate any improvement in breathing. For example, the patient may be asked to rank the improvement on a scale of zero to five, five corresponding to significant improvement and zero corresponding to no discernible improvement.

If the breathing has not improved by a discernible amount, then at operation 325, if there are other nose clip configurations, at operation 330, different nose clip configurations may be selected and the operations from 310 may repeat until either the different nose clip configurations are exhausted or a nose clip producing a discernible improvement is found. For example, the second nose clip may be evaluated followed by the third nose clip.

If at operation 315, a nose clip producing a discernible improvement is found, the clinician may determine that a procedure for correcting the disorder is available and may recommend performing the procedure to the patient. For example, if the first nose clip having the narrowest width, W1, improves nasal breathing, then a minimally invasive device placed along the sidewall may provide improved nasal breathing. If the second nose clip having the next narrowest width, W1, improves nasal breathing, then an alar batten graft or lateral crural strut graft would likely provide improved nasal breathing. If the third nose clip having the next largest width, W1, improves nasal breathing, then repositioning of the lateral crura with lateral crural strut grafts and an alar batten graft may provide improved breathing.

Thus, the nose clip and diagnostic method provide a reliable way of selecting an appropriate treatment to lateral wall collapse and nasal valve collapse/obstruction in the nose. This, in turn, reduces the likelihood of unnecessary and/or ineffective procedures being performed on the patient.

While the diagnostic method and nose clip used in the method for selecting treatment for lateral wall weakness and nasal valve collapse/obstruction have been described with reference to certain embodiments, it will be understood by those skilled in the art that various changes may be made and equivalents may be substituted without departing from the scope. Therefore, it is intended that the present apparatus and method of using the apparatus not be limited to the particular embodiment disclosed, but that the method and system include all embodiments falling within the scope of the appended claims.

I claim:

1. A method for selecting an appropriate treatment for a patient with nasal valve lateral wall collapse or nasal valve collapse/obstruction, the method comprising:
forming a plurality of nose clips from a semi-rigid material, each nose clip having an outer support section configured to overlay a contour of an outside surface of a nose; an inner support section configured to be placed inside the nasal cavity of the nose and to push the lateral wall of the nasal cavity outward towards the outer support section; and a bridge section that connects the outer support to the inner support section;
configuring a first nose clip of the plurality to provide a first amount of support between the inner and outer support section;
configuring a second nose clip of the plurality to provide a second amount of support between the inner and outer support section that is greater than the first level amount of force;
inserting the first nose clip into a nasal cavity of the patient;
determining whether insertion of the first nose clip sufficiently reduces nasal valve lateral wall collapse in the patient;
if the first nose clip sufficiently reduces nasal valve lateral wall collapse, determining that a first treatment will effectively reduce nasal valve lateral wall collapse in the patient;
if the reduction in nasal valve lateral wall collapse is insufficient, inserting the second nose clip into the nasal cavity of the patient;
determining whether insertion of the second nose clip sufficiently reduces nasal valve lateral wall collapse; and
if the second nose clip sufficiently reduces nasal valve lateral wall collapse, determining that a second treatment will effectively reduce nasal valve lateral wall collapse in the patient.

2. The method according to claim 1, wherein configuring the first and second nose clips to provide first and second different amounts of support comprises forming the first and second nose clips from a ribbon shaped material, wherein a width of the first nose clip is smaller than a width of the second nose clip to thereby reduce a relative amount of support provided by the first nose clip as compared to the second nose clip.

3. The method according to claim 1, wherein configuring the first and second nose clips to provide first and second different amounts of support comprises:
setting a distance between the outer and inner support sections of the first nose clip to a first distance; and
setting a distance between the outer and inner support sections of the second nose clip to a second distance that is less than the first distance.

4. The method according to claim 1, wherein the plurality of nose clips includes a first set of nose clips configured to provide the first amount of support between the inner and outer support sections, and a second set of nose clips configured to provide the second amount of support between the inner and outer support sections, wherein the first and second sets of nose clips include nose clips having different lengths as measured from the bridge to a top of the inner support section.

5. The method according to claim 1, wherein the different lengths include lengths of: 1.2 cm, 1.5 cm and 2 cm.

6. The method according to claim 1, have substantially the same shape.

7. The method according to claim 1, wherein determining whether insertion of the first nose clip sufficiently reduces nasal valve lateral wall collapse in the patient comprises:
receiving, from the patient, a numerical rating that reflects an amount of improvement to breathing due to insertion of the first nose clip, where a rating of zero indicates no improvement and numbers greater than zero indicate successive amounts of greater improvement; and
when the rating is below a predetermined value, determining that the improvement provided by the first nose clip is insufficient.

8. A diagnostic kit for diagnosing lateral wall weakness and nasal valve collapse/obstruction of a patient's nose comprising:
a plurality of nose clips formed from a semi-rigid material, each nose clip having:
an outer support section configured to overlay a contour of an outside surface of a nose;
an inner support section configured to be placed inside the nasal cavity of the nose and to push the lateral wall of the nasal cavity outward towards the outer support section; and
a bridge section that connects the outer support to the inner support section,
wherein a first nose clip of the plurality is configured to provide a first amount of support between the inner and outer support sections;
wherein a second nose clip of the plurality is configured to provide a second amount of support between the inner and outer support sections that is greater than the first amount of support,
wherein each nose clip of the plurality is associated with one of a plurality medical procedures for correcting lateral wall weakness, and during diagnoses, nose clips of the plurality are inserted into the patient's nose to identify a nose clip of the plurality that provides a discernable improvement in breathing, and thereby determining a medical procedure for the correcting lateral wall weakness of the patient's nose.

9. The diagnostic kit according to claim 8, wherein the plurality of nose clips are formed from a ribbon shaped material, wherein a width of the ribbon shaped material of the first nose clip is smaller than a width of the ribbon shaped material of the second nose clip to thereby reduce a relative amount of support provided by the first nose clip as compared to the second nose clip.

10. The diagnostic kit according to claim 8, wherein a distance between the outer and inner support sections of the first nose clip is set to a first distance; and a distance between the outer and inner support sections of a second nose clip is set to a second distance that is less than the first distance.

11. The diagnostic kit according to claim 8, wherein the plurality of nose clips includes a first set of nose clips configured to provide the first amount of support between the inner and outer support sections, and a second set of nose clips configured to provide the second amount of support between the inner and outer support sections, wherein the first and second sets of nose clips include nose clips having different lengths as measured from the bridge section to a top of the inner support section.

12. The diagnostic kit according to claim 8, wherein the different lengths include lengths of: 1.2 cm, 1.5 cm and 2 cm.

13. The diagnostic kit according to claim 8, wherein the patient rates an amount of improvement to breathing due to insertion of the first nose clip, wherein when the rating is at or above a predetermined value, a procedure associated with the nose clip is determined to be sufficient to correct the lateral wall weakness and nasal valve collapse/obstruction.

14. The diagnostic kit according to claim 13, wherein when the rating is below the predetermined value, the procedure associated with the nose clip is determined to be insufficient to correct the lateral wall weakness and nasal valve collapse/obstruction.
